(19) United States
(12) Patent Application Publication
(10) Pub. No.: US 2012/0071876 A1
(43) Pub. Date: Mar. 22, 2012

(54) MICROFRACTURE AWL

(76) Inventors: E. Jordan STOLL, BOULDER, CO (US); Jeremy S. JAMES, WESTMINSTER, CO (US)

(21) Appl. No.: 12/885,235

(22) Filed: Sep. 17, 2010

(51) Int. Cl. A61B 17/16; A61B 17/56

(52) U.S. Cl. 606/79; 606/96

(57) ABSTRACT

A microfracture awl includes an articulating portion that enables the microfracture awl to strike a subchondral bone plate at a precise desired angle to prevent scuffing or undue scraping of the targeted subchondral bone plate. The articulating portion of the microfracture awl is received through a guide sleeve. A proximal end of the articulating portion is rigid and connects to a base. The articulating portion is placed through a guide sleeve and the base resides in a handle attached to the sleeve. The guide sleeve has a curved distal end. The practitioner strikes the rigid base of the microfracture awl and thereby transmits a force to the articulating portion of the awl however, the force is not transmitted to the guide sleeve or handle. Accordingly, the articulating portion of the awl may smoothly travel through the guide sleeve at the angle defined by the curved distal end of the guide sleeve, thereby angularly positioning the tip of the awl with the targeted bone plate. The articulating portion includes a plurality of articulating members configured in a series of ball and socket configurations. A method includes selection of a guide sleeve with the desired curvature and impacting the awl a number of times at the targeted area to produce a desired effect with respect to the release of bone marrow cells to induce growth of fibrocartilage tissue.

### FIELD OF THE INVENTION

The invention relates to an apparatus and method for repairing cartilage in damaged joints, and more particularly, an apparatus and method for conducting microfracture on subchondral bone to repair articular cartilage.

### BACKGROUND OF THE INVENTION

Articular cartilage is an avascular tissue made of chondrocytes arranged in an extra cellular matrix. Articular cartilage allows for surfaces of abutting bone structures to articulate smoothly with a minimal amount of friction. The cartilage also acts as a means to absorb various forces such as tensile, compressive, and shear forces that are experienced at the joint. Failure of articular cartilage can be a debilitating condition that is very difficult to successfully treat, and results in significant pain for the patient. Because of the inability of articular cartilage to repair as quickly as other tissues such as muscle, damaged articular cartilage often continues to degenerate and progress to conditions such as osteoarthritis.

One procedure developed to induce cartilage growth in joints is the use of a microfracture awl to create holes in the subchondral bone plate of damaged joints where the cartilage loss or deterioration exists. By creating holes in the bone at the correct depth, bone marrow cells are released to the surface of the joint and form a pool. These released immature or bone marrow stem cells are pluripotential. That is, these stem cells can then differentiate into either bone or cartilage, depending upon the local environment in which the cells have been released. This bone marrow stimulating procedure therefore results in a fibrin clot formation and the migration of the stem cells from the bone marrow to the location of the defective cartilage. Over time, the released stem cells develop into the fibrocartilage that fills the damaged surface area of the joint to provide relief to pain and arthritis symptoms.

Typically, the microfracture awl has a distal pointed tip, and the proximal base of the awl is manually struck with a mallet. The distal tip forms holes in the subchondral bone plate. Care is made to not penetrate the bone too deeply and to otherwise damage the subchondral plate. In a successful procedure, the holes penetrate a vascularization zone and stimulate the formation of the fibrin clot containing the pluripotential stem cells. Depending up the number of holes, the clot fills the defective joint area and matures into the fibrocartilage.

Microfracturing techniques have proven to be successful procedures for producing fibrocartilage tissue and to therefore repair defective cartilage. However, there are a number of distinct disadvantages with the use of known microfracture awls.

Current microfracture awls are made of a solid construction in which the awl has a tip formed at a fixed angle, such as thirty, forty-five or ninety degrees. It is generally desirable to strike the bone of the joint perpendicular to the bone surface in order to most effectively penetrate the bone without scuffing or shifting of the awl tip that otherwise can shatter or fragment pieces of the bone. Particularly for smaller joints, it is even more important to contact the bone surface at the perpendicular orientation to prevent bone damage beyond the intended penetration. However, existing one-piece awls with the various fixed angles do not allow for creation of a perpendicular force as the mallet strikes the tip of the awl. The curved tip of the awl inherently results in a considerable amount of force being transmitted in a more parallel orientation to the bone surface as opposed the desired perpendicular orientation. Further, even when a practitioner selects an awl tip with a lesser angled tip, it is often difficult to orient the awl tip in the desired perpendicular arrangement because of the small spaces between abutting bone surfaces in the joint. Therefore, whether the practitioner chooses an awl tip with a greater angle or lesser angle, the awl has a tendency to scuff the joint and shift as the mallet imparts the impacting force. Even with a very sharp tipped awl, migration of the tip of the awl upon impact with the bone can be a significant problem.

Another type of microfracture device currently available is one that drills holes into the bone plate. While this device may help to avoid damage to the bone structure since the drill will presumably not shift during use, this device is not capable of providing the desired perpendicular orientation of the shaft of the drill bit and the bone surface. Specifically, the drill bit is a linear and fixed element, and therefore the drill bit cannot be oriented to approach many bone surfaces in the desired perpendicular orientation. Additionally, the use of a drilling device may result in thermal necrosis to the bone and to the bone marrow stem cells caused by the temperature of the drill.

Therefore, there is a need to provide a microfracture awl that can be oriented to transfer a force perpendicular to the bone surface in which the mallet does not have to also be oriented perpendicular to the targeted bone surface. There is also a need to provide an awl that has the capability to transfer the perpendicular force within a wide arrangement of impact angles, depending upon the particular subchondral bone plate encountered. There is also a need to provide an awl with these characteristics in which the awl is of a robust construction, a simple design and cost-effective design, and easily sterilizable. There is yet a further need for a device to avoid the potential for thermal necrosis and damage to the very cells necessary for the fibrocartilage overgrowth of the damaged area.

### SUMMARY OF THE INVENTION

The invention is directed to a microfracture awl and method for repairing defects in articular cartilage. The awl includes an articulating distal portion comprising a plurality of interconnected articulating members that enables the awl to be configured in an infinite number of angular positions with respect to the targeted bone structure, yet the awl can still transfer a specifically directed force to the bone. The articulating members are characterized by a group of interconnected ball members arranged in a chain or series in which each articulating member is capable of rotating or articulating with respect to the articulating members connected at opposite ends. The distal end of the articulating portion includes a sharpened contacting tip or awl tip that penetrates the subchondral bone plate.

The articulating portion has a proximal end connected to a rigid shaft. A proximal end of the rigid shaft in turn connects to a base. A guide sleeve is provided to receive the working end or awl tip. The proximal end of the guide sleeve attaches to a handle. The articulating distal portion of the awl is inserted through the guide sleeve, and the base is housed within the handle. The awl tip of the articulating portion protrudes beyond the distal end of the sleeve and is exposed for contacting the targeted bone plate. The proximal end of the base may include a widened area in the form of a cap that is contacted by a force transferring object such as a mallet.

The distal end of the guide sleeve is provided with the necessary curvature that allows the practitioner to insert the awl within the confined space of the joint to contact the targeted bone structure and to appropriately align the awl tip for contacting the bone structure. Initially, the awl tip of the articulating portion is fully contained within the guide sleeve. Upon impact by the mallet against the cap of the awl, the articulating portion of the apparatus protrudes beyond the distal end of the guide sleeve, and penetrates the targeted bone structure at the desired perpendicular orientation. The articulating portion of the awl travels smoothly through the guide sleeve. Therefore, the force of the impact provided by the practitioner in swinging the mallet to contact the cap in turn causes the force to be transferred through the base, shaft and articulating portion of the awl, while the handle and guide sleeve remain in place to guide the working end of the awl. The guide sleeve and handle do not receive the force from the mallet that could otherwise cause displacement of the awl tip away from the desired perpendicular orientation. A number of microfractures are created by repeated contact of the awl tip against the bone plate.

The articulating portion represents a structure that may change its orientation by articulating movement to follow the path of the guide sleeve, yet is able to withstand a compression force, such as applied by a mallet, without the articulating portion itself slipping or shifting that may otherwise change the orientation with respect to how the awl tip approaches the targeted bone structure, yet the articulating portion also effectively transfers the compression force from the awl tip of the articulating portion to the bone.

The present invention can be provided in the form of a kit in which a number of guide sleeves are provided with differing angled ends. However, because of the articulating capability of the awl, a single articulating portion is all that is required for multiple procedures.

The device of the present invention can be reusable or disposable, allowing for cost of manufacturing and sterilization parameters to be satisfied. For disposable devices, it is desirable to select a cost effective materials for use, which may include for example, aluminum or plastics. For reusable devices, it is desirable to choose materials conducive to repeated heat sterilization, particularly for the articulating distal portion of the awl, such as stainless steel.

According to a method of the present invention, microfracture surgery is performed on subchondral bone in which the practitioner selects a guide sleeve with the desired angular end. The articulating portion of the awl is inserted through the guide sleeve, and the base is seated within the handle. In one embodiment, a retention pin is inserted through a slot formed in the handle and through an opening in the base to slidably retain the base within the handle. The practitioner locates the distal end of the guide sleeve against the targeted bone, and strikes the cap with a blunt object such as a mallet. The force of the impact is transmitted through the base, shaft and articulating portion, while the guide sleeve and handle remains substantially stationary without receiving the force from the mallet. The articulating portion is pushed through the guide sleeve and makes contact with the targeted bone structure at the desired chosen angle. The guide sleeve is then shifted to the next location for which to create a microfracture, and the practitioner strikes the cap again to make the microfracture. The bone is penetrated to a sufficient depth to induce the formation of a pool of the bone marrow tissue that subsequently grows into the fibrocartilage.

Other features and advantages of the apparatus and method of the invention will become apparent with the review of the following detailed description taken in conjunction with the drawings.

### BRIEF DESCRIPTION OF THE DRAWINGS

FIG. 1 is an exploded perspective view of the microfracture awl;

FIG. 2 is a perspective view of the base, shaft, and articulating portion comprising a first subcombination;

FIG. 3 is a perspective view of the guide sleeve and handle comprising a second subcombination, and showing the first subcombination attached wherein the articulating portion and shaft are inserted through the guide sleeve and the base is secured within the handle;

FIG. 4 is an enlarged fragmentary cross-section illustrating the articulating portion of the awl received in the guide sleeve;

FIG. 5 is a greatly enlarged fragmentary cross-sectional view of an awl tip;

FIG. 6 is a greatly enlarged perspective view of an articulating member;

FIG. 7 is a greatly enlarged fragmentary cross-sectional view of a pair of articulating members connected to one another;

FIG. 8 is an enlarged fragmentary cross-sectional view of the articulating portion of the awl received within the guide sleeve, showing the articulating capability of the articulating members enabling the articulating portion to smoothly traverse through the angled distal end of the guide sleeve; and

FIG. 9 is a perspective view of a joint in which the microfracture awl is positioned for contacting a targeted subchondral bone plate within the joint.

### DETAILED DESCRIPTION

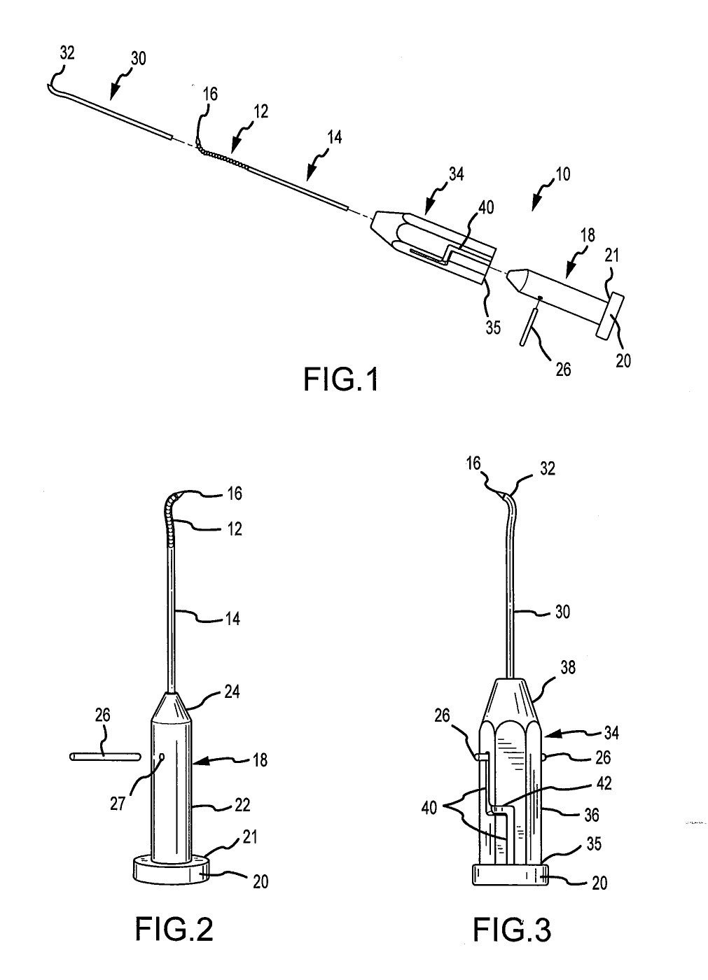

Referring to FIGS. 1-3, the microfracture awl 10 is illustrated. The microfracture awl 10 includes two subcombinations. The first subcombination comprises an articulating portion 12 of the awl, a rigid shaft 14, and a base 18. The proximal end of the articulating portion 12 connects to a distal end of the rigid shaft 14. The rigid shaft 14 attaches at its proximal end to a distal end of the base 18. The proximal end of the base 18 may include an enlarged portion or cap 20 that is struck by a mallet (not shown) to transfer force through the awl to the bone. The base 18 is shown as having a substantially cylindrical shaped body 22 and a distal converging portion 24. A retention pin opening 27 is formed transversely through the body 22 and receives a retention pin 26. The awl tip 16 of the articulating portion 12 makes contact with and is driven through a targeted subchondral bone plate as explained below.

The second subcombination comprises a guide sleeve 30 having a diameter to slidably receive the articulating portion 12 and shaft 14. The guide sleeve has a curved distal end 32. The curvature of the distal end 32 determines the angular change in the direction of force. The proximal end of the guide sleeve connects to a handle 34 held by the practitioner. The handle has an opening sized to receive the base 18. Referring specifically to FIG. 3, the handle 34 may include a beveled exterior 36 and a converging portion 38. The beveled exterior 36 assists in holding the handle, but it shall be understood that the handle may adopt other features to assist in grasping such as a handle 34 with a knurled finish. A slot 40 is formed on opposite sides of the handle to receive the retention pin 26 as shown. The slot 40 has an offset 42 between opposing ends of the slot 40. The base 18 is able to travel a limited distance distally and longitudinally within the opening of the handle. The base 18 can be removed from within the handle, but either requires the base to be rotated so the pin 26 can traverse the offset 42, or the pin 26 can be removed. Thus, the offset 42 serves to hold the base 18 within the handle 34.

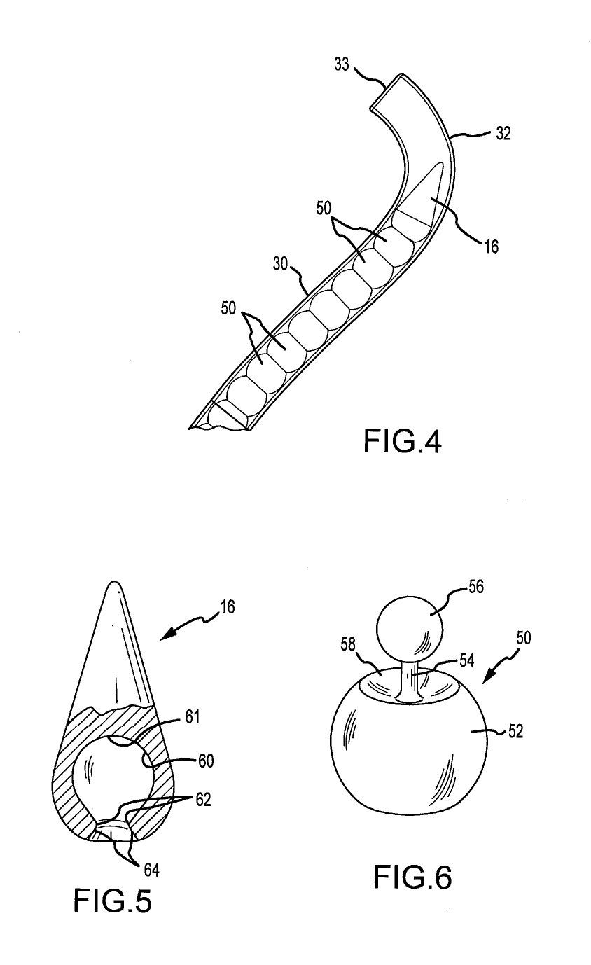

Referring to FIG. 4, the articulating portion 12 is shown as being received within the guide sleeve 30. The articulating portion 12 comprises a plurality of independently rotatable and interconnected articulating members. The diameter of the sleeve 30 can be selected to allow the articulating portion to smoothly travel through the sleeve without binding and without appreciable lateral shifting of the members 50 that may otherwise create undue frictional resistance. While the Figures show a rigid shaft 14 and articulating portion 12, it is also contemplated that the shaft 14 can be eliminated in favor of only an articulating portion that attaches directly to the base 18.

Referring to FIGS. 5-7, details are provided for the construction of the articulating portion 12 and the awl tip 16. Referring first to FIG. 5, the tip 16 is illustrated in cross-section to expose a spherical shaped socket 60. The socket 60 communicates with an opening defined by a converging surface 62 as shown. This opening in turn transitions into a larger opening towards the exterior surface of the tip 16 as defined by diverging surface 64. Referring to FIGS. 6 and 7, construction details of the articulating members 50 are shown. The articulating members are connected to one another in series or a chain configuration. Each of the articulating members 50 has a spherical shaped body 52, a male portion and a female portion. The male portion includes, a stem 54 that protrudes from the body 52 and a connector 56 attached to the distal end of the stem. Preferably, the connector 56 is also spherical. The stem 54 is centered within a spherical shaped recess 58 formed on the outer surface of the body 52. The female portion of a member 50 is of a similar construction as the socket 60 of the tip 16. Accordingly as shown in FIG. 7, each of the ball members 50 can be described as having a spherical socket 60, a converging surface 62 defining an opening, and a diverging surface 64 defining a larger opening closer to the exterior surface of the member 50. As shown in the FIG. 7, the male portion of one articulating member is located within the female portion of the adjacent articulating member. The diameter of the connector 56 is larger than the diameter of the smaller opening defined by the converging surface 62, thereby preventing disconnection of the adjacent ball members. The diameter of the connector 56 and the length of the stem 54 are sized to prevent the connector 56 from making contact with the opposing end of the interior wall 61 of the socket 60 when the adjacent ball members are compressed towards one another as discussed below.

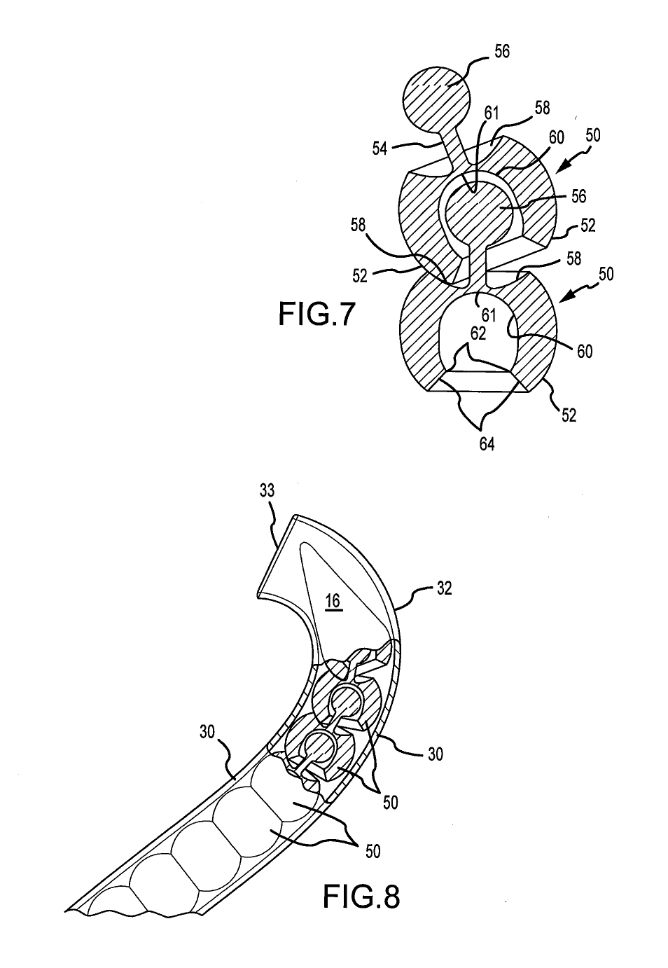

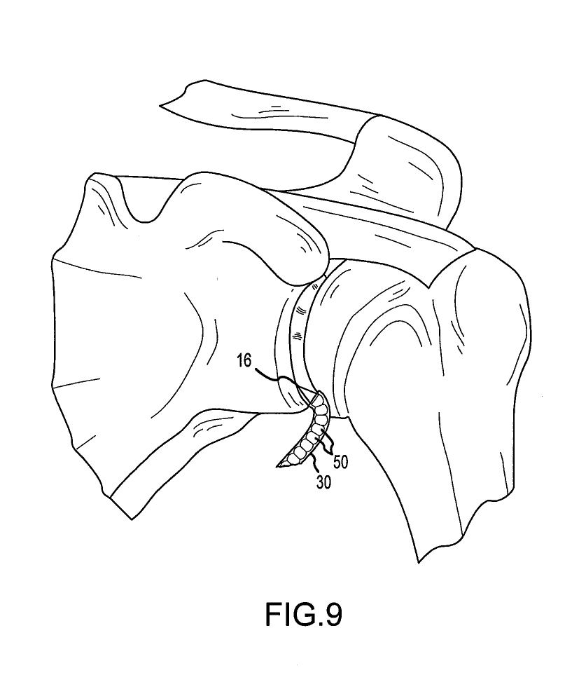

Referring to FIG. 8, the articulating portion 12 is illustrated as being further inserted through the guide sleeve 30 and this figure shows the articulating capability of the articulating portion in moving through the curved distal end 32. The construction of the articulating members 50 allows them to articulate or rotate in order to follow the curved shaped of the distal end 32 of the guide sleeve. The exterior spherical body 52 of one member 50 is received within the spherical recess 58 of the adjacent member 50, thus creating a ball and socket interaction between adjacent members 50 that enables the articulating portion 12 of the awl to follow the curved path of the guide sleeve 30. When the practitioner is prepared to create a microfracture after positioning the distal end 33 of the guide sleeve at the targeted bone location, the practitioner will use a device such as a mallet to strike the cap 20. Initially, the articulating portion 12 is placed in the position such as shown in FIG. 4 in which the tip 16 does not protrude beyond the distal opening 33. Once the practitioner strikes the cap, the articulating portion travels through the guide sleeve as shown in FIG. 8, and the tip 16 protrudes beyond the distal opening 33 to contact and penetrate the bone. The combined length of the base 18, shaft 14 and articulating portion 12 is precisely determined with respect to the combined length of the guide sleeve 30 and handle 34 such that the facing surface 21 of the cap 20 does not make contact with the proximal facing end 35 of the handle. Otherwise, the impact of the mallet against the cap 20 could also simultaneously transfer force to the handle 34 and guide sleeve 30, which would reduce the amount of force directly transferred to the articulating portion and would therefore reduce the force direction change capability of the articulating portion traveling through the guide sleeve. The combined length of the articulating portion, shaft and base subcombination is also taken into account also with respect to the depth to which the practitioner wishes to penetrate the subchondral bone plate to ensure that there is no contact between the surface 21 of the cap and the proximal end 35 of the handle 34. In a typical procedure, a depth of penetration for the microfracture should be between about 2 to 6 mm. Preferably, only a portion of the tip 16 protrudes beyond the distal opening 33 during use. Retaining a portion of the awl tip 16 within the sleeve 30 stabilizes the tip so that it does not inadvertently rotate and slip upon transfer of the force to the bone.

When the microfracture awl receives the force from a mallet, each of the articulating members 50 make contact with one another by the chain of spherical recesses 58 and the abutting exterior surfaces of the spherical bodies 52. Accordingly, a small gap is maintained between the connectors 56 and the interior wall 61 and there is no contact of the connectors 56 against the opposing surfaces of the interior walls 61, which could otherwise break or disfigure the stems 54. The connectors 56 make contact with other portions of the interior walls 61, for example when two members 50 are offset in travelling through the curved end 32 of the sleeve, but the connectors 56 are not compressed in this manner. Thus, the unique construction of the articulating portion 12 enables repeated use without damaging the connections between the articulating members.

While a particular construction is illustrated in a preferred embodiment with respect to the articulating portion 12, it shall be understood that the articulating portion more broadly represents a structure that may change its orientation by articulating movement to follow the path of the guide sleeve, yet is able to withstand a compression force, such as applied by a mallet, without the articulating portion itself slipping or shifting that may otherwise change the orientation with respect to how the awl tip approaches the targeted bone structure. Additionally, the articulating portion effectively transfers the compression force from the awl tip of the articulating portion to the bone.

According to the method of the invention, a practitioner chooses a guide sleeve 30 having the desired curvature in the distal curved end 32. The articulating portion 12 is inserted through the guide sleeve 30, and the base 18 is seated within the handle 34. The retention pin 26 is placed through the slot 40 and through the retention pin opening 27. The practitioner then places the distal end 33 of the guide sleeve 30 at the targeted located on the bone structure, and then applies a force against the cap 20 to transfer force to the tip 16 of the articulating portion 12 to thereby penetrate the bone. The location of the guide sleeve is moved to the next targeted location, and the practitioner may then again strike the cap to create the next microfracture. The process is repeated a number of times to provide the desired pattern and number of microfractures to adequately pool the bone marrow cells over the damaged cartilage location. If a practitioner wishes to change the particular angle at which the awl tip 16 strikes the subchondral bone plate, the practitioner has the option of obtaining another handle and guide sleeve subcombination with the desired curvature for the distal end 32. Thus, other guide sleeves may be selected from the kit to change the angular orientation of the guide sleeve that allows the practitioner to best position the awl for contact with the targeted bone plate, yet the practitioner is still able to maintain the substantially perpendicular orientation of the tip 16 with respect to the targeted bone plate.

After the microfracturing process is completed and the release of bone marrow is adequate, all of the instruments are removed from the joint. Preferably, the joint may be evacuated from fluid except for the fluid that pools intra-articularly, since this fluid is rich in the bone marrow cells which should be allowed to form and stabilize where the microfractures are located. The microfracture technique also produces a relatively rough surface that enhances the ability of the clot to adhere to the surrounding bone.

After the tip 16 has struck the bone, there can be some recoil of the awl causing proximal movement of the base 18. However as shown in the preferred embodiment, the base 18 is prevented from being withdrawn from the handle due to the offset 42 in the slot 40. Therefore, recoil of the awl still allows the practitioner to use just one hand in operating the awl since the base will not be separated from the handle without also manually twisting the base 18 or removing the pin 26.

FIG. 9 illustrates the distal end 32 of the guide sleeve 30 placed within a joint and against a targeted subchondral bone plate. In the example of FIG. 9, the illustrated joint is the glenoid cavity, also showing the adjacent humeral head. As shown, the curvature in the distal end 32 of the guide sleeve 30 enables the awl tip 16 to be oriented at an optimum angle for contacting the glenoid. The practitioner can comfortably strike the cap 20 of the base 18 causing transfer of the force to the subchondral bone plate in a precise directional manner. Although FIG. 9 shows use of the apparatus with one example of a joint, it shall be understood that the invention is not limited to use with any particular joint or bone surface. There are a number of other joints for which the microfracture awl is ideally suited. For example, the tibial plafond and talus, femoral head and acetabullum, and tibial plateau are other bone structures that can be treated with the method and apparatus of the present invention.

The present invention has been described with respect to one or more preferred embodiments to include an apparatus and method. However, it should be understood that various other changes and modifications to this apparatus and method may be made within the scope of the claims appended hereto. Accordingly, it is also appropriate that the pending claims be construed in a manner that is consistent with the broad teachings of the summary and detailed description.

What is claimed is:

1. A microfracture awl comprising;
an articulating awl subcombination including (i) an articulating portion having proximal and distal ends, (ii) a shaft having proximal and distal ends, a distal end of the shaft connected to the proximal end of the articulating portion, and (iii) a base connected to a proximal end of the shaft;
a guide subcombination for receiving the articulating awl subcombination including (i) a guide sleeve having proximal and distal ends, and (ii) a handle, the proximal end of the guide sleeve connected to a distal end of the handle;
said articulating portion including a plurality of articulating members placed in series, each of the articulating members including a male portion and a female portion, the male portions of each of the articulating members inserted within the female portions of adjacent articulating members.

2. An awl, as claimed in claim 1, wherein:
said distal end of said articulating portion includes an awl tip for contacting a targeted subchondral bone plate, and said awl tip including a female portion for receiving a male portion of an adjacent articulating member.

3. An awl, as claimed in claim 1, wherein:
said distal end said guide sleeve is curved.

4. An awl, as claimed in claim 1, wherein:
said base is received in said handle, said handle includes a slot, and a retaining pin is placed through an opening formed in said body and through said slot thereby retaining said base within said handle.

5. An awl, as claimed in claim 1, wherein:
each of said male portions includes a curved recess, a stem extending from the articulating member, and a connector attached to the stem, wherein the connector is received within the adjacent female portion of the adjacent articulating member.

6. An apparatus, as claimed in claim 1, wherein:
each of said female portions includes a socket forming a spherical cavity, and an opening communicating with said socket.

7. An awl, as claimed in claim 1, wherein:
each of said male portions includes a curved recess, a stem extending from the articulating member, and a connector attached to the stem, wherein the connector is received within the adjacent female portion of the adjacent articulating member;
each of said female portions includes a socket forming a spherical cavity, and an opening communicating with said socket; and
wherein said connector is received within the socket.

8. A method for performing microfracture, said method comprising:
providing an articulating awl subcombination including (i) an articulating portion having a proximal end and a distal awl tip, (ii) a shaft having proximal and distal ends, a distal end of the shaft connected to the proximal end of the articulating portion, and (iii) a base connected to a proximal end of the shaft;
providing a guide subcombination for receiving the microfracture awl, the guide subcombination including (i) a guide sleeve having a proximal end, a curved distal end, and (ii) a handle, the proximal end of the guide sleeve connected to a distal end of the handle;
placing the distal end of the guide sleeve on a targeted subchondral bone plate;
striking the proximal end of the base to transfer a force through the articulating awl subcombination;
displacing the articulating portion through the guide sleeve so that the awl tip of the articulating portion changes direction along the curved distal end of the guide sleeve, and the awl tip protrudes beyond the distal end of the guide sleeve;
contacting and penetrating the subchondral bone plate a desired depth with the awl tip.

9. A microfracture awl comprising;
an articulating awl subcombination including an articulating portion having proximal and distal ends, and a base connected to a proximal end of the articulating portion;
a guide subcombination for receiving the articulating awl subcombination including (i) a guide sleeve having proximal and distal ends, and (ii) a handle, the proximal end of the guide sleeve connected to a distal end of the handle;
said articulating portion including a plurality of articulating members placed in series; and
wherein said articulating portion is inserted through said guide sleeve and said base is received within an opening of said handle.

10. A microfracture awl subcombination comprising:
an articulating portion having proximal and distal ends, and a plurality of articulating members placed in series, each of said articulating members being rotatable with respect to an adjacent articulating member;
a base connected to a proximal end of the articulating portion; and
an awl tip connected to the distal end of the articulating portion.
